(19) United States
(12) Patent Application Publication
(10) Pub. No.: US 2005/0234331 A1
(43) Pub. Date: Oct. 20, 2005

(54) Method, apparatus and program for obtaining differential image

(75) Inventor: Tomonari Sendai, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/086,439

(22) Filed: Mar. 23, 2005

(30) Foreign Application Priority Data
Mar. 23, 2004 (JP) 085110/2004
Sep. 2, 2004 (JP) 255637/2004

(51) Int. Cl. A61B 5/05

(52) U.S. Cl. 600/425

(57) ABSTRACT

Detection of the difference between images of the same subject obtained at different times is performed effectively. The cardiac phases of the subject are detected, and a current image is obtained through X-raying the subject at the time when the detected cardiac phase corresponds to that of the past image. Thereafter, a differential image is obtained from the current and past images by obtaining the difference between them.

### BACKGROUND OF THE INVENTION

1. Field of the Invention

The present invention relates to a method for obtaining images through X-raying a subject and a differential image from the X-ray images obtained. More specifically, the present invention relates to a differential image obtaining method and apparatus for obtaining a differential chest image. It also relates to a program for causing a computer to execute the differential image obtaining method.

2. Description of the Related Art

In order to monitor the time course of changes in a diseased area through radiographic images, a so-called temporally subtraction technology is proposed as described, for example, in the non-patent document “Digital image subtraction of temporally sequential chest images for detection of interval change”, A. Kano, K. Doi, H. MacMahon, D. Hassell and M. L. Ginger, Med. Phys. 21(3), March 1994, pp. 453-461. The technology is used to generate a differential image from temporally sequential radiographic images of a diseased area in order to highlight the portion that has changed during a certain time interval for aiding the medical staff in identifying the changed portion. The simultaneous observation of the differential image obtained in the manner described above together with the temporally sequential radiographic images greatly facilitates the diagnosis of the diseased area.

A system for obtaining a differential image from two images of the same subject obtained at different times by using the temporally subtraction technology is proposed as described, for example, in Japanese Unexamined Patent Publication No. 2002-158923. The method allows a pale shadow, such as early-stage lung cancer and the like, to be detected by highlighting the difference between the two images obtained at different times and indicating the progress of the affected area.

In making comparisons between past and current X-ray images, there may be a case where the comparison is made between the past X-ray image obtained at a time when the heart was contracted and the current X-ray image obtained at a time when the heart was dilated. In this case, the comparison is made between the image of dilated lung fields and that of the deformed lung fields by the pressure of the heart, and the difference between the two images may not be identified easily. Consequently, a system for obtaining X-ray images is proposed as described, for example, in Japanese Unexamined Patent Publication No. 2003-250790, in which X-ray images are obtained invariably at a time when the heart is contracted by obtaining the cardiac cycle through measuring contractions and dilations of the heart using a measuring device, such as an electrocardiograph or a plethysmograph.

Even if the temporally subtraction technology is applied to chest imaging as in Japanese Unexamined Patent Publication No. 2002-158923, a small pale shadow, such as a candidate of lung cancer or the like located in the vicinity of the heart, may not be identified due to the motion artifacts arising from the cardiac beat or respiration.

On the other hand, if X-ray images are obtained at a time when the heart is actually contracted, as described in Japanese Unexamined Patent Publication No. 2003-250790, the comparison between the X-ray images obtained at different times may become easier, but the visual inspection has its own limit for identifying a small pale shadow, such as a shadow of lung cancer or the like, even if it is present in the current X-ray image.

### SUMMARY OF THE INVENTION

The present invention has been developed in view of the circumstances described above, and it is an object of the present invention to provide a differential image obtaining method and apparatus capable of identifying the difference between the X-ray images obtained at different times efficiently. It is a further object of the present invention to provide a program for causing a computer to execute the differential image obtaining method.

A differential image obtaining method according to the present invention is a method, in which a current image is obtained through X-raying, and a differential image is obtained by obtaining the difference between the current image and a past image provided in advance through X-raying, the method comprising the steps of:
detecting cardiac cycle phases of a subject;
obtaining the current image through X-raying at a time when the cardiac cycle phase detected by the cardiac cycle phase detecting step corresponds to the cardiac cycle phase of the past image; and
obtaining the differential image from the current and past images.

A differential image obtaining apparatus according to the present invention is an apparatus, in which a current image is obtained through X-raying, and a differential image is obtained by obtaining the difference between the current image and a past image provided in advance through X-raying, the apparatus comprising:
a detecting means for detecting cardiac cycle phases of a subject;
a current image obtaining means for obtaining the current image through X-raying at a time when the cardiac cycle phase detected by the detecting means corresponds to the cardiac cycle phase of the past image; and
a differential image obtaining means for obtaining the differential image from the current and past images.

The current image obtaining means may further comprise a scattered radiation removing grid; a grid moving means for moving the gird; and a grid control means for controlling the grid moving means such that the grid reaches the maximum traveling speed when the current image is obtained.

Another differential image obtaining apparatus according to the present invention comprises:
a past image storing means for storing a past image obtained through X-raying the chest of a subject and cardiac cycle information that indicates the cardiac cycle phase of the subject at the time when the past image was obtained;
a radiographing means for X-raying a subject;
a cardiac cycle phase detecting means for detecting cardiac cycle phases of the subject;
a control means for controlling the radiographing means such that a current image is obtained through X-raying the chest of the subject at a time when the cardiac cycle phase detected by the cardiac cycle phase detecting means corresponds to the cardiac cycle phase of the subject at the time when the past image was obtained; and
a differential image obtaining means for obtaining a differential image from the past and current images.

A program according to the present invention is a program for causing a computer to execute:
a past image storing step for storing a past image obtained through X-raying the chest of a subject, and cardiac cycle information that indicates the cardiac cycle phase of the subject at the time when the past image was obtained;
a control step for controlling a radiographing means such that a current image is obtained through X-raying the chest of the subject at a time when the cardiac cycle phase detected by a cardiac cycle phase detecting means for detecting cardiac cycle phases of the subject corresponds to the cardiac cycle phase of the subject at the time when the past image was obtained; and
a differential image obtaining step for obtaining a differential image from the past and current images.

The referent of “cardiac cycle phase” as used herein means a position in a cycle of the cyclic movement of cardiac contractions and dilations.

As for the “cardiac cycle phase detecting means”, for example, an electrocardiograph, plethysmograph, or the like may be used.

Still another differential image obtaining apparatus according to the present invention comprises:
a past image storing means for storing a past image obtained through X-raying the chest of a subject, cardiac cycle information that indicates the cardiac cycle phase of the subject at the time when the past image was obtained, and respiration information that indicates the respiration phase of the subject at the time when the past image was obtained;
a radiographing means for X-raying a subject;
a cardiac cycle phase detecting means for detecting cardiac cycle phases of the subject;
a respiration phase detecting means for detecting respiration phases of the subject;
a control means for controlling the radiographing means such that a current image is obtained through X-raying the chest of the subject at a time when the cardiac cycle phase detected by the cardiac cycle phase detecting means corresponds to the cardiac cycle phase of the subject at the time when the past image was obtained, and the respiration phase detected by the respiration phase detecting means corresponds to the respiration phase of the subject at the time when the past image was obtained; and
a differential image obtaining means for obtaining a differential image from the past and current images.

The referent of “respiration phase” as used herein means a position in a cycle of the cyclic movement of inhalations and exhalations of the lungs.

As for the “respiration phase detecting means”, for example, a spirometer, pulmometer, respiration monitoring belt, or a device for detecting the respiration phase by monitoring the respiration with a photo camera may be used.

Yet another differential image obtaining apparatus according to the present invention comprises:
a past image storing means for storing a past image obtained through X-raying the chest of a subject, and cardiac cycle information that indicates the cardiac cycle phase of the subject at the time when the past image was obtained;
a radiographing means for X-raying a subject;
a cardiac cycle phase detecting means for detecting the cardiac cycle phase of the subject;
a respiration phase detecting means for detecting the respiration phase of the subject;
a control means for controlling the radiographing means such that a current image is obtained through X-raying the chest of the subject at a time when the cardiac cycle phase detected by the cardiac cycle phase detecting means corresponds to the cardiac cycle phase of the subject at the time when the past image was obtained; and
a correcting means for correcting at least either the past or current image such that the past and current images have the identical respiration phase based on the respiration phase of the past image, and the respiration phase detected by the respiration detecting means at the time when the current image was obtained; and
a differential image obtaining means for obtaining a differential image from the past and current images having the identical respiration phase.

The radiographing means may further comprise a scattered radiation removing grid, and a grid moving means for moving the gird. The control means may be configured to further control the grid moving means such that the grid reaches the maximum traveling speed when the current image is obtained.

Another differential image obtaining apparatus according to the present invention comprises:
a past image storing means for storing a past image obtained through X-raying the chest of a subject, and cardiac cycle information that indicates the cardiac cycle phase of the subject at the time when the past image was obtained;
a current image storing means for storing a current image obtained through X-raying the chest of a subject and cardiac cycle information that indicates the cardiac cycle phase of the subject at the time when the current image was obtained;
a correcting means for correcting at least either the past or current image such that the past and current images have the identical cardiac cycle phase based on the cardiac cycle phase at the time when the past image was obtained and the cardiac cycle phase at the time when the current image was obtained; and
a differential image obtaining means for obtaining a differential image from the past and current images having the identical cardiac cycle phase.

Another differential image obtaining method according to the present invention comprises:
a past image storing step for storing a past image obtained through X-raying the chest of a subject, and cardiac cycle information that indicates the cardiac cycle phase of the subject at the time when the past image was obtained;
a current image storing step for storing a current image obtained through X-raying the chest of the subject and cardiac cycle information that indicates the cardiac cycle phase of the subject at the time when the current image was obtained;
a correcting step for correcting at least either the past or current image such that the past and current images have the identical cardiac cycle phase based on the cardiac cycle phase at the time when the past image was obtained, and the cardiac cycle phase at the time when the current image was obtained; and
a differential image obtaining step for obtaining a differential image from the past and current images having the identical cardiac cycle phase.

Another program according to the present invention is a program for causing a computer to execute:
a past image storing step for storing a past image obtained through X-raying the chest of a subject, and cardiac cycle information that indicates the cardiac cycle phase of the subject at the time when the past image was obtained;
a current image storing step for storing a current image obtained through X-raying the chest of a subject, and cardiac cycle information that indicates the cardiac cycle phase of the subject at the time when the current image was obtained;
a correcting step for correcting at least either the past or current image such that the past and current images have the identical cardiac cycle phase based on the cardiac cycle phase at the time when the past image was obtained, and the cardiac cycle phase at the time when the current image was obtained; and
a differential image obtaining step for obtaining a differential image from the past and current images having the identical cardiac cycle phase.

The “current image” may be an image obtained in advance as long as it has been obtained after the “past image”.

Still another differential image obtaining apparatus according to the present invention comprises:
a past image storing means for storing a past image obtained through X-raying the chest of a subject, cardiac cycle information that indicates the cardiac cycle phase of the subject at the time when the past image was obtained, and respiration information that indicates the respiration phase of the subject at the time when the past image was obtained;
a current image storing means for storing a current image obtained through X-raying the chest of the subject, cardiac cycle information that indicates the cardiac cycle phase of the subject at the time when the current image was obtained, and respiration information that indicates the respiration phase of the subject at the time when the current image was obtained;
a correcting means for correcting at least either the past or current image such that the past and current images have the identical cardiac cycle and respiration phases based on the cardiac cycle and respiration phases at the time when the past image was obtained, and the cardiac cycle and respiration phases at the time when the current image was obtained; and
a differential image obtaining means for obtaining a differential image from the past and current images having the identical cardiac cycle and respiration phases.

Preferably, the differential image obtaining means further comprises an aligning means for aligning the thoraces between the past and current images.

According to the present invention, when X-raying the chest of a subject with the subject holding the breath, cardiac cycle phases of the subject are detected, and a current image is obtained through X-raying the subject at a time when the detected cardiac cycle phase corresponds to that of the past image. Then, a differential image is obtained from the current and past images, so that a pale shadow, such as lung cancer and the like, may be highlighted.

Further, when X-raying the chest of a subject, respiration phases as well as the cardiac cycle phases of the subject are detected, and a current image is obtained through X-raying the subject at a time when the detected cardiac cycle and respiration phases correspond to those of the past image, and a differential image is obtained from the current and past images. Thus, even while the subject is breathing voluntarily without holding the breath, a current image having the identical cardiac cycle and respiration phases as those of the past image may be obtained, and a pale shadow, such as lung cancer or the like, may be highlighted.

Still further, if a current image is obtained with the subject breathing voluntarily without hold the breath and thus the past and current images have only the identical cardiac cycle phase, either the past or current image is corrected such that the images have also the identical respiration phase. Thereafter, a differential image is obtained from the past and current images, so that a pale shadow, such as lung cancer or the like, may be highlighted.

Further, in cases where the chest image of the subject is obtained with the subject holding the breath, either the past or current image is corrected such that the past and current images have the identical cardiac cycle phase before the differential image is obtained. Thus, a pale shadow, such as lung cancer or the like, may be highlighted using images obtained at different cardiac cycle phases.

By obtaining a differential image after either the past or current image has been corrected such that the past and current images have the identical respiration and cardiac cycle phases, a pale shadow, such as lung cancer or the like, may be highlighted using images obtained with the subject breathing voluntarily without holding the breath and having different cardiac cycle phases.

Further, by performing the aligning operation before obtaining the difference between the past and current images, a precise location of lung cancer may be identified.

Still further, by using a scattered radiation removing grid when obtaining the current image, which is controlled such that it reaches the maximum traveling speed at the time of imaging, the scattered radiation may be prevented, and at the same time an image without the grid pattern superimposed thereon may be obtained.

### BRIEF DESCRIPTION OF THE DRAWINGS

FIG. 1 is a schematic block diagram of the differential image obtaining apparatus according to a first embodiment of the present invention.

FIG. 2 is a schematic block diagram of the differential image obtaining apparatus according to a second embodiment of the present invention.

FIG. 3 is a schematic block diagram of the differential image obtaining apparatus according to a third embodiment of the present invention.

FIG. 4A is a drawing illustrating example templates for different cardiac cycle phases.

FIG. 4B is a drawing illustrating example templates for different cardiac cycle phases.

FIG. 5 is a drawing illustrating respective regions within a thorax.

FIG. 6 is a drawing illustrating the lung fields that vary in accordance with the cardiac motion.

FIG. 7A is a drawing illustrating the correction method for matching cardiac cycle phases.

FIG. 7B is a drawing illustrating the correction method for matching cardiac cycle phases.

FIG. 7C is a drawing illustrating the correction method for matching cardiac cycle phases.

FIG. 8 is a schematic block diagram of the differential image obtaining apparatus according to a fourth embodiment of the present invention.

FIG. 9A is a drawing illustrating example templates for different respiration phases.

FIG. 9B is a drawing illustrating example templates for different respiration phases.

FIG. 10A is a drawing illustrating the correction method for matching respiration phases.

FIG. 10B is a drawing illustrating the correction method for matching respiration phases.

FIG. 10C is a drawing illustrating the correction method for matching respiration phases.

FIG. 11 is a schematic block diagram of the differential image obtaining apparatus according to a fifth embodiment of the present invention.

FIG. 12 is a drawing illustrating the configuration for obtaining an image using a scattered radiation removing grid (part 1).

FIG. 13 is a drawing illustrating the configuration for obtaining an image using a scattered radiation removing grid (part 2).

### DESCRIPTION OF THE PREFERRED EMBODIMENTS

Hereinafter, the differential image obtaining apparatus according to a first embodiment will be described with reference to the accompanying drawings.

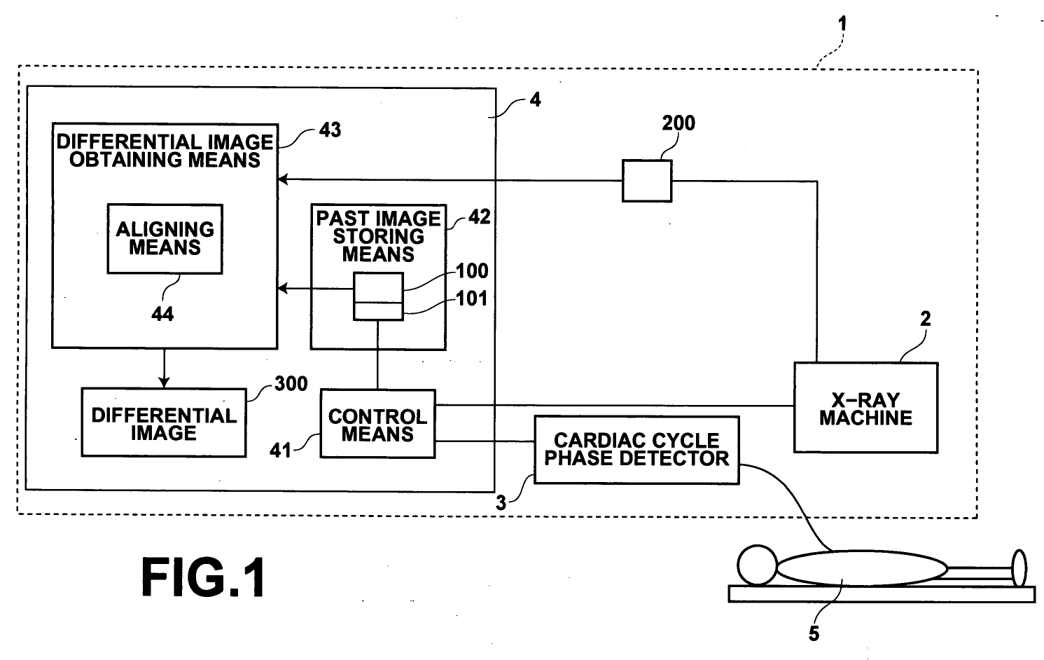

Now, reference is made to FIG. 1. As shown in FIG. 1, a differential image obtaining apparatus 1 of the present invention comprises: an X-ray machine (radiographing means) 2, such as computed radiography (CR) or the like, for X-raying a subject 5; a cardiac cycle phase detector (cardiac cycle phase detecting means) 3 for detecting cardiac cycle phases of the subject 5; and a computer 4 for controlling the X-ray machine 2 for the timing of imaging.

The computer 4 has a control means 41 for controlling the X-ray machine 2 for the timing of imaging; a past image storing means 42 for storing a past image 100 obtained by the X-ray machine 2; and a differential image obtaining means 43 for obtaining a differential image 300 from the past image 100 and a current image 200. The differential image obtaining means 43 has an aligning means 44 for aligning the thoraces between the past and current images.

When an X-ray generation signal for instructing the X-ray machine 2 to X-ray the subject is inputted from the control means 41, the X-ray machine 2 irradiates X-rays to the subject 5 from an X-ray generating source. The X-rays irradiated from the X-ray generating source are passed through the chest of the subject 5, and detected by a planar X-ray detector that detects the X-ray map. The planar X-ray detector converts the detected X-rays to electrical signals, which are then converted to digital signals through A/D conversion. The digital signals are then stored in a memory within the X-ray machine 2 as an image, which will be transferred to the computer 4 as required.

The past image storing means 42 is a mass storage device, such as a hard disk provided for the computer 4, or a memory within the computer. It receives a chest X-ray of the subject 5 from the X-ray machine 2, and stores it as the past image 100. Alternatively, the chest X-ray of the subject 5 may be stored in a portable storage medium, such as a DVD, which is then stored in the past image storing means 42 from the portable storage medium. Further, the images of the subject 5 may be stored in a file server or the like which is connected to a network, and a relevant image of the subject 5 among the plurality of images of the subject 5 is retrieved and read into the past image storing means 42.

The past image storing means 42 stores the past image 100, and cardiac cycle information 101 that indicates the cardiac cycle phase of the subject 5 at the time when the past image 100 was obtained. The cardiac cycle information 101 may be stored in the file of the past image 100 as accompanying information, or it may be stored and managed in a separate file, as long as it is related to the past image 100. For example, the file of the past image 100 may be stored in a hard disk, while the cardiac cycle information 101 may be stored in a memory within a computer.

The control means 41 is connected to the cardiac cycle phase detector 3, and receives cardiac cycle phases of the subject 5 in real time. It outputs the X-ray generation signal to the X-ray machine 2 when the cardiac cycle phase received from the subject 5 becomes a desired timing for instructing the X-ray machine 2 (current image obtaining means) to X-ray the subject 5.

The cardiac cycle phase detector 3 is an electrocardiograph, plethysmograph, or the like for detecting cardiac cycle phases of the subject 5. It detects cardiac cycle phases of the subject 5 arising from the cardiac contractions and dilations as analog signals, which are then converted to digital signals through A/D conversion, and sent to the control means 41 in real time.

Hereinafter, in the first embodiment, the operation flow in which the current image 200 of a chest X-ray of the subject 5 is obtained at the time when the cardiac cycle phase of the subject 5 corresponds to that of the past image 100 will be described in detail.

In general, when a chest X-ray of a subject is obtained, the imaging is performed with the subject holding the breath after taking a deep breath to dilate the lungs. But the cardiac cycle can not be ceased. Consequently, in order to obtain the current image 200 having the identical cardiac cycle phase to that of the past image 100, the cardiac cycle phase at the time when the past image was obtained, and the cardiac cycle phase at the time when the current image 200 is obtained need to be detected. Therefore, the cardiac cycle phase detector 3 for detecting the cardiac cycle phases of the subject 5, such as an electrocardiograph, plethysmograph, or the like, is attached thereto when these images are obtained.

When X-raying the chest of the subject 5 using an electrocardiograph to detect the cardiac cycle phases, if the sensor of the electrocardiograph is attached to the chest region, it interferes the X-raying. Therefore, the sensor is preferably attached to the region other than the chest region, such as the arm or the leg. There are various types of plethysmographs including photoelectric types, piezoelectric types, pulse oximeters, accelerated plethysmographs. Of these, the use of the pulse oximeter which is attached to the fingertip, earlobe or the like, or the accelerated plethysmograph which is attached to the fingertip allows the X-raying of the chest of the subject 5 without interference.

The cardiac cycle phases of the subject 5 detected by the electrocardiograph or plethysmograph are digitized and inputted to the control means 41 in real time. The control means 41 stores the cardiac cycle phase at the time when the X-ray generation signal was outputted to the X-ray machine 2 to cause it to obtain the past image 100 in the past image storing means 42 as the cardiac cycle information 101, and receives the chest X-ray obtained by the X-ray machine 2 to store it in the past image storing means 42 as the past image 100.

In order to obtain the current image 200 having the identical cardiac cycle phase to that of the past image 100, the control means 41 detects the cardiac cycle phases of the subject 5 in real time, and outputs the X-ray generation signal to the X-ray machine 2 for instructing it to obtain the current image 200 when the detected cardiac cycle phase of the subject 5 corresponds to that included in the cardiac cycle information 101 of the past image 100 stored in the past image storing means 42.

Here, there may be a time lag between a time when the cardiac was actually contracted and the time when the cardiac contraction is detected by the electrocardiograph or plethysmograph. Therefore, such a time lag, which may arise when the sensor of the electrocardiograph is attached to the arm or when the plethysmograph is attached to the fingertip, is measured in advance, and the actual cardiac cycle phase is determined according to the time lag measured in advance. In addition, there is also a time lag between the time when the X-ray generation signal is received and the time when X-rays will be actually irradiated from the X-ray generating source. Therefore, the X-ray generation signal is outputted to the X-ray machine 2 taking into account this time lag.

The current image 200 having the identical cardiac cycle phase to that of the past image 100 obtained in the manner described above is transferred to the computer 4, where a differential image 300 is generated by the differential image obtaining means 43 by obtaining the difference between the past image 100 stored in the past image storing means 42 and the current image 200. In generating the differential image 300, if the location of the chest region of the subject 5 in the current image 200 differs from that in the past image 100, the artifacts arising from misalignment may result. Consequently, the thoraces between the past image 100 and current image 200 are aligned by the aligning means 44 before the differential image 300 is generated.

The aligning means 44 employs, for example, the automatic thorax detecting method, in which the thoraces are detected from the past image 100 and current image 200 through template matching using templates having substantially analogous shapes to the contours of the standard cardiac shape (Reference is made to Japanese Unexamined Patent Publication No. 2002-109548 and Japanese Unexamined Patent Publication No. 2003-006661 proposed by the applicant for detail), and affine transformation is performed on the extracted thoraces so that they correspond to each other, thereby the thoraces between the past image 100 and current image 200 are aligned.

As has been described hereinbefore, the current image is obtained when the cardiac cycle phase of the subject corresponds to that of the past image. Further, the differential image is obtained from the past and current images after the aligning operation has been performed between them. This allows a pale shadow, such as a shadow of lung cancer or the like appeared in the vicinity of the heart, may be highlighted without faded by the influence of the cardiac movement (motion artifacts).

Hereinafter, the differential image obtaining apparatus according to a second embodiment will be described. The apparatus according to the present embodiment obtains a differential image from the past and current images of the subject 5 which have been obtained with the subject 5 breathing voluntarily without holding the breath. In the present embodiment, components identical to those used in the first embodiment are given the same reference numerals and will not be elaborated upon further here.

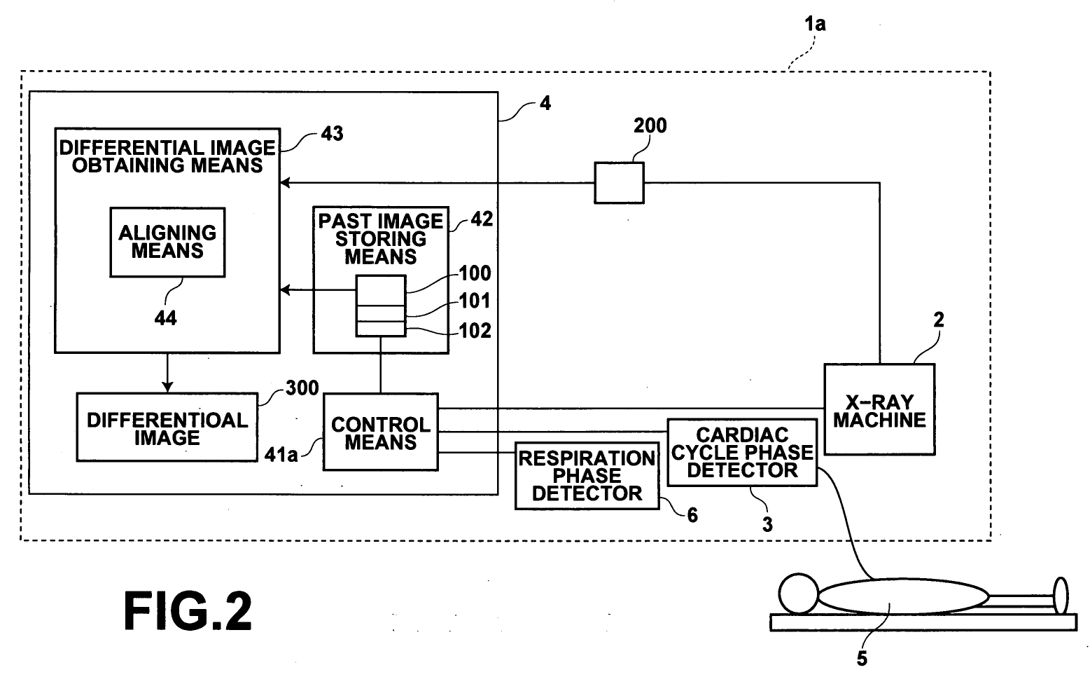

Now, reference is made to FIG. 2. As shown in FIG. 2, a differential image obtaining apparatus 1a of the present invention comprises: the X-ray machine (radiographing means) 2, such as computed radiography (CR) or the like, for X-raying the subject 5; the cardiac cycle phase detector (cardiac cycle phase detecting means) 3 for detecting cardiac cycle phases of the subject 5; a respiration phase detector (respiration phase detecting means) 6 for detecting respiration phases of the subject 5; and the computer 4 for controlling the X-ray machine 2 for the timing of imaging.

The computer 4 has a control means 41a for controlling the X-ray machine 2 for the timing of imaging; a past image storing means 42 for storing the past image 100 obtained by the X-ray machine 2; and the differential image obtaining means 43 for obtaining the differential image 300 from the past image 100 and the current image 200. The differential image obtaining means 43 has the aligning means 44 for aligning the thoraces between the past and current images.

The past image storing means 42 stores not only the cardiac cycle information 101 that indicates the cardiac cycle phase of the subject 5 at the time when the past image 100 was obtained, but also respiration information 102 that indicates the respiration phase of the subject 5 at the time when the past image 100 was obtained in addition to the past image 100.

The control means 41a is connected to the cardiac cycle phase detector 3 and respiration phase detector 6, and receives cardiac cycle and respiration phases of the subject 5 in real time. It outputs the X-ray generation signal to the X-ray machine 2 when both of the cardiac and respiration phases received from the subject 5 become desired timing for instructing the X-ray machine 2 (current image obtaining means) to X-ray the subject 5.

The respiration phase detector 6 is a device for detecting respiration phases of the subject. More specifically, the respiration cycle of the subject may be detected, for example, with a spirometer, pulmometer, respiration monitoring belt, or a device for detecting the respiration phase by monitoring the respiration with a photo camera. The analog signals detected by the respiration phase detector 6 are converted to digital signals through A/D conversion, and sent to the control means 41a in real time.

When obtaining the past image 100 and current image 200, the cardiac cycle phase detector 3 and respiration phase detector 6 are attached to the subject 5, so that the current image 200 and past image 100 have the identical cardiac and respiration phases.

The control means 41a receives cardiac and respiration phases of the subject 5 respectively from the cardiac cycle phase detector 3 and respiration detector 6 connected thereto in real time. It outputs the X-ray generation signal to the X-ray machine 2 to cause it to obtain the current image 200 when the detected cardiac cycle phase of the subject 5 corresponds to that included in the cardiac cycle information 101 of the past image 100, and detected respiration phase of the subject 5 corresponds to that included in the respiration information 102 of the past image 100.

As in the detection of the cardiac cycle phase described above, there may be a time lag between the actual and detected respiration phases. Preferably, therefore, such a time lag is measured in advance, and the actual respiration phase is determined according to the time lag measured in advance.

Thereafter, as in the first embodiment, the thoraces between the past image 100 and current image 200 are aligned by the aligning means 44 before a differential image 300 is generated by the differential image obtaining means 43 by obtaining the difference between the past image 100 and current image 200.

In the second embodiment described above, the respiration phase detector 6 is used for detecting respiration phases of the subject. But, an alternative arrangement may be made without using the respiration phase detector 6, in which the chest region of the subject 5 is scanned by the X-ray machine 2 with a low radiation dosage to obtain chest X-rays, which are checked in real time, and the respiration phase obtained in this manner is sent to the control means 41a.

As has been described in detail hereinbefore, the current image is obtained when both the cardiac and respiration phases correspond to those of the past image. Further, the differential image is obtained from the past and current images after the aligning operation has been performed between them. This allows a pale shadow, such as a shadow of lung cancer, may be highlighted even when the chest X-rays are obtained with the subject breathing voluntarily.

In the first and second embodiments, the differential image obtaining apparatus comprises an X-ray machine and a computer provided separately. But an alternative arrangement may be made in which each of the means within the computer may be provided in the X-ray machine.

Hereinafter, the differential image obtaining apparatus according to a third embodiment of the present invention will be described. The apparatus according to the present embodiment obtains a differential image from the past and current images having different cardiac cycle phases after either the past or current image has been corrected. In the present embodiment, chest X-rays are obtained with the subject holding the breath after taking a deep breath to dilate the lungs. Further, in the present embodiment, components identical to those used in the first and second embodiments are given the same reference numerals and will not be elaborated upon further here.

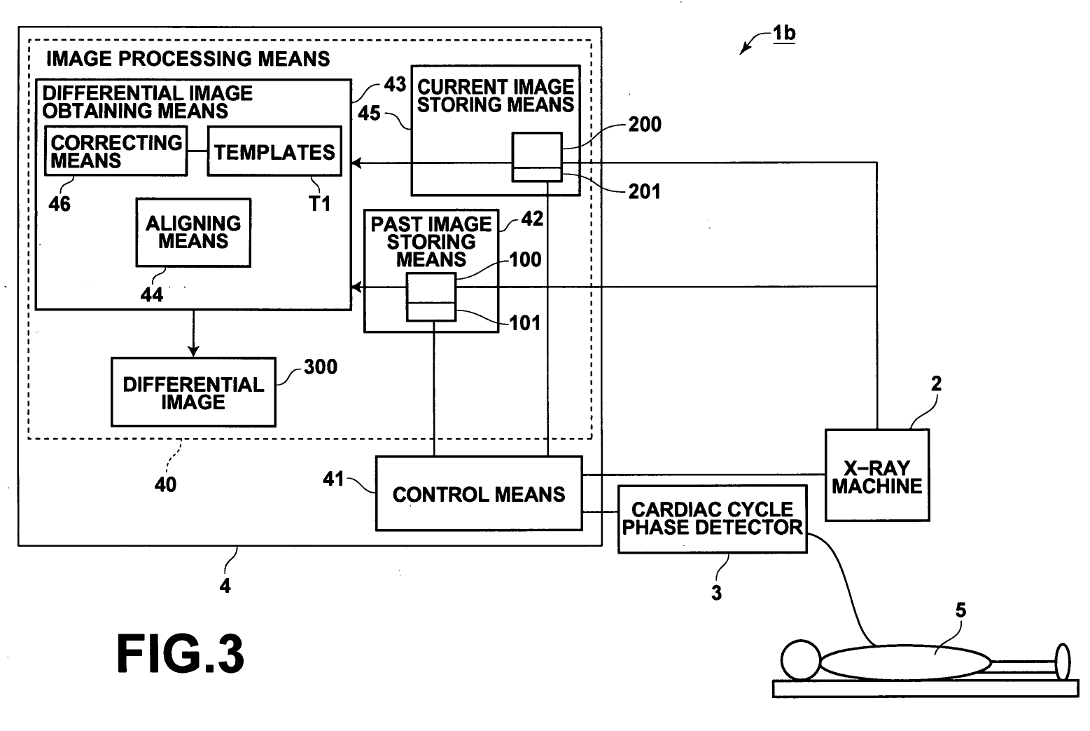

Now reference is made to FIG. 3. As shown in FIG. 3, a differential image obtaining apparatus 1b of the present invention comprises: the X-ray machine (radiographing means) 2, such as computed radiography (CR) or the like, for X-raying the subject 5; the cardiac cycle phase detector (cardiac cycle phase detecting means) 3 for detecting cardiac cycle phases of the subject 5; and the computer 4 for controlling the X-ray machine 2 for the timing of imaging.

The computer 4 has the control means 41 for controlling the X-ray machine 2 for the timing of imaging, and an image processing means 40 for obtaining a differential image from the images obtained by the X-ray machine 2.

The image processing means 40 has the past image storing means 42 for storing the past image 100 obtained by the X-ray machine 2; a current image storing means 45 for storing the current image 200 obtained by the X-ray machine 2; and the differential image obtaining means 43 for obtaining the differential image 300. The differential image obtaining means 43 has a correcting means 46 for correcting the past image such that the past and current images have the identical cardiac cycle phase, and the aligning means 44 for aligning the thoraces between the past image 100 and current image 200.

The past image storing means 42 and current image storing means are mass storage devices, such as a hard disk provided for the computer 4 or a memory within the computer. They receive chest X-rays of the subject 5 from the X-ray machine 2, and store them as the past image 100 and current image 200 respectively.

The past image storing means 42 stores the past image 100, and cardiac cycle information 101 that indicates the cardiac cycle phase of the subject 5 at the time when the past image 100 was obtained. The current image storing means 45 stores the current image 200, and cardiac cycle information 201 that indicates the cardiac cycle phase of the subject 5 at the time when the current image 100 was obtained. Cardiac cycle phases of the subject 5 are inputted to the control means 41 from the cardiac cycle phase detector 3 in real time, and the cardiac cycle phases at the time when the past and current images were obtained are stored as the cardiac cycle information 101 and 102 respectively.

Alternatively, the cardiac cycle information 101 and 201 obtained at the time when the respective images were obtained as well as the past image 100 and current image 200 may be stored first in a portable storage medium, such as a DVD, which are then read into the past image storing means 42 and current image storing means respectively from the storage medium. Further, images and the cardiac cycle information of subjects may be stored in a file server or the like connected to a network, and the past image 100 and current image 200 with the respective cardiac cycle information of a relevant subject are retrieved and read into the past image storing means 42 and current image storing means 45 respectively.

The shape of the lung fields in the vicinity of the heart changes according to the cardiac cycle phases. If imaging is performed at a time when the heart is contracted, a chest image with dilated lung fields is obtained, and if it is performed at a time when the heart is dilated, a chest image with pressed lung fields in the vicinity of the heart due to the pressure of the heart is obtained. Thus, when obtaining the difference between the past image 100 and current image 200, if images having different cardiac cycle phases are used, a pale shadow, such as lung cancer or the like located in the vicinity of the heart, may not be highlighted. Consequently, if either the past image 100 or current image 200 is corrected such that they have the identical cardiac cycle phase, and the differential image 300 is generated by obtaining the difference between the past image 100 and current image 200 having the identical cardiac cycle phase, then a pale shadow, such as lung cancer or the like, may be highlighted.

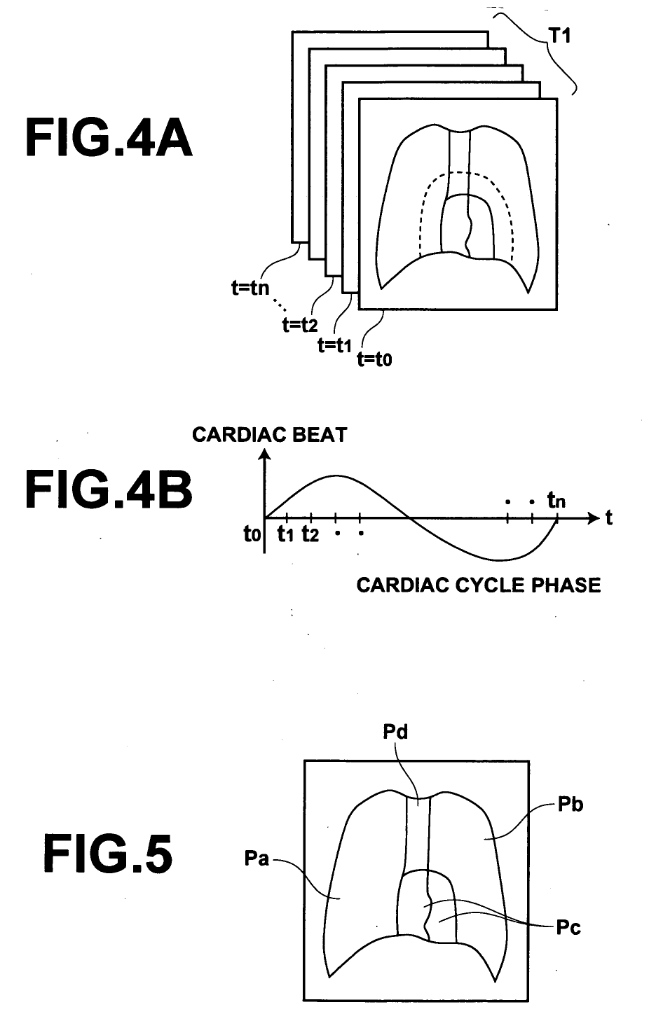

Consequently, a series of X-ray images of the subject are obtained with a low radiation dosage in the initial stage of monitoring the time course of changes in a diseased area of the subject in order to enable the subsequent corrections of the cardiac cycle phases of the images obtained through X-raying the subject. This produces a plurality of images having different cardiac cycle phases, and templates T1 as shown in FIG. 4A, each corresponding to each of the cardiac cycle phases as shown in FIG. 4B are created. The templates T1 are provided at sufficiently short intervals so that a template having the identical cardiac cycle phase to that of an image subsequently obtained is always available.

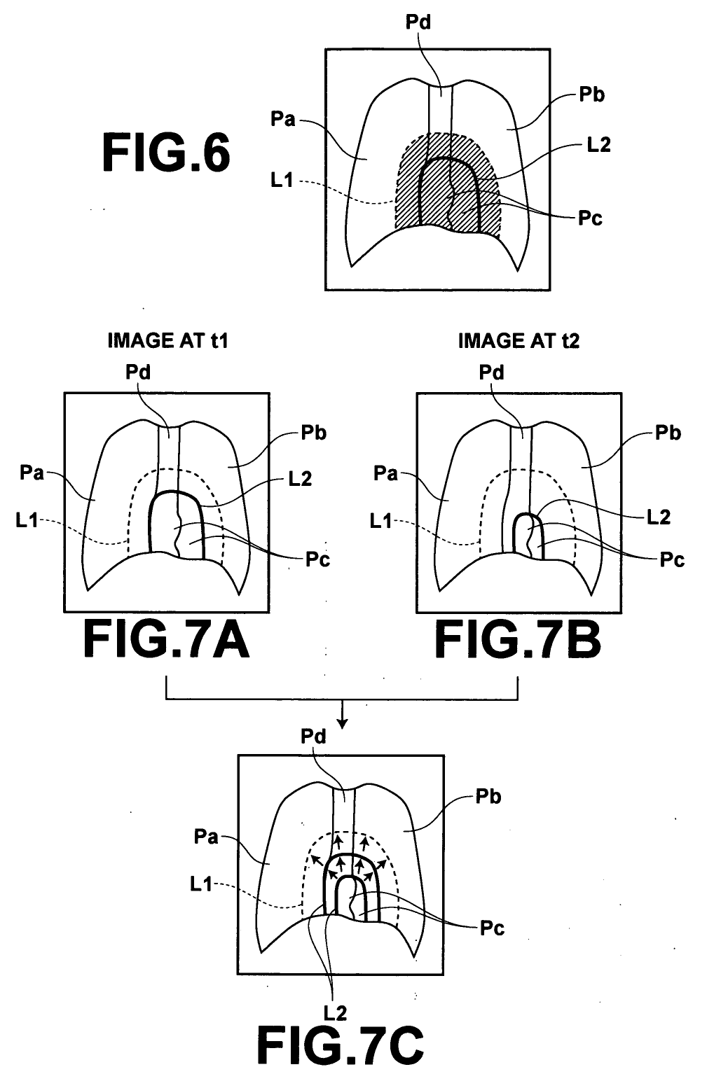

The lung fields in the vicinity of the heart will change largely in accordance with the cardiac movement, but the regions away from the heart will not be influenced by the cardiac movement. Consequently, each of the regions, such as the lung field regions (Pa, Pb), mediastinal region (Pd), cardiac region (Pc), and the like as shown in FIG. 5, is extracted from each of the templates T1 (reference is made to Japanese Unexamined Patent Publication No. 2003-006661 for detail). Then, based on the data obtained empirically from the images, a region extending from the cardiac region to a certain area of the lung field regions is defined as a variable region that varies according to the cardiac cycle phase (shaded area in FIG. 6, which may be, for example, approximately twice the area of the cardiac region). The shape of the lung fields at the circumference L1 (dotted line in FIG. 6) of the variable region remains the same between images having different cardiac cycle phases, that is, the amount of variation in the shape of the lung fields between the images is 0. On the other hand, the shape of the lung fields at the circumference L2 (bold line in FIG. 6) of the cardiac region varies the most between the images having different cardiac cycle phases, and the amount of variation in the shape of the lung fields between the images having different cardiac cycle phases decreases gradually from the circumference L2 of the cardiac region to the circumference L1 of the variable region.

For example, as shown in FIGS. 7A and 7B, when transforming the image at a cardiac cycle phase t1 (FIG. 7A) into the image at a cardiac cycle phase t2 (FIG. 7B), pixels of the circumference L2 of the cardiac region at the cardiac cycle phase t1 are warped to the locations that correspond to the circumference L2 of the cardiac region at the cardiac cycle phase t2. Then, the pixels contained in the variable region at the cardiac cycle phase t1 are warped such that the amount of variation in the pixel position decreases gradually from the circumference L2 of the cardiac region to the circumference L1 of the variable region, with the pixels of the circumference L1 of the variable region kept at the original positions (FIG. 7C).

The correcting means 46 first extracts each of the regions, such as the lung field regions (Pa, Pb), mediastinal region (Pd), cardiac region (Pc), and the like, from the current image 200. Further, it extracts the variable region of the current image 200 using the templates T1 of the cardiac cycle phases of the current image 200. Then, it warps pixels present in the variable region of the extracted current image 200 to the positions of the template T1 having a cardiac cycle phase that corresponds to that of the past image 100. In the manner described above, the current image 200 is corrected to have a cardiac cycle phase that corresponds to that of the past image 100.

As in the embodiments described above, the differential image obtaining means 43 performs the aligning operation (through the aligning means 44) between the current image 200, which has been corrected to have the cardiac cycle phase that corresponds to that of the past image 100, and the past image 100 before obtaining the differential image 300.

In the embodiment described above, the current image is corrected to have a cardiac cycle phase that corresponds to that of the past image. It will be appreciated that the past image may be corrected to have a cardiac cycle phase that corresponds to that of the current image.

As has been described hereinbefore, in the present embodiment, either the past or current image is corrected so that both images have the identical cardiac cycle phase. Then, the aligning operation is performed between the past and current images before obtaining the differential image. This allows a pale shadow, such as lung cancer or the like appeared in the vicinity of the heart, to be highlighted without faded by the influence of the cardiac movement (motion artifacts).

Hereinafter, the differential image obtaining apparatus according to a fourth embodiment of the present invention will be described. In the present embodiment, as in the second embodiment, X-raying is performed with the subject 5 breathing voluntarily without holding the breath. But it is performed when the cardiac cycle phase of the subject 5 corresponds to that of the past image. In the present embodiment, components identical to those used in the embodiments described above are given the same reference numerals and will not be elaborated upon further here.

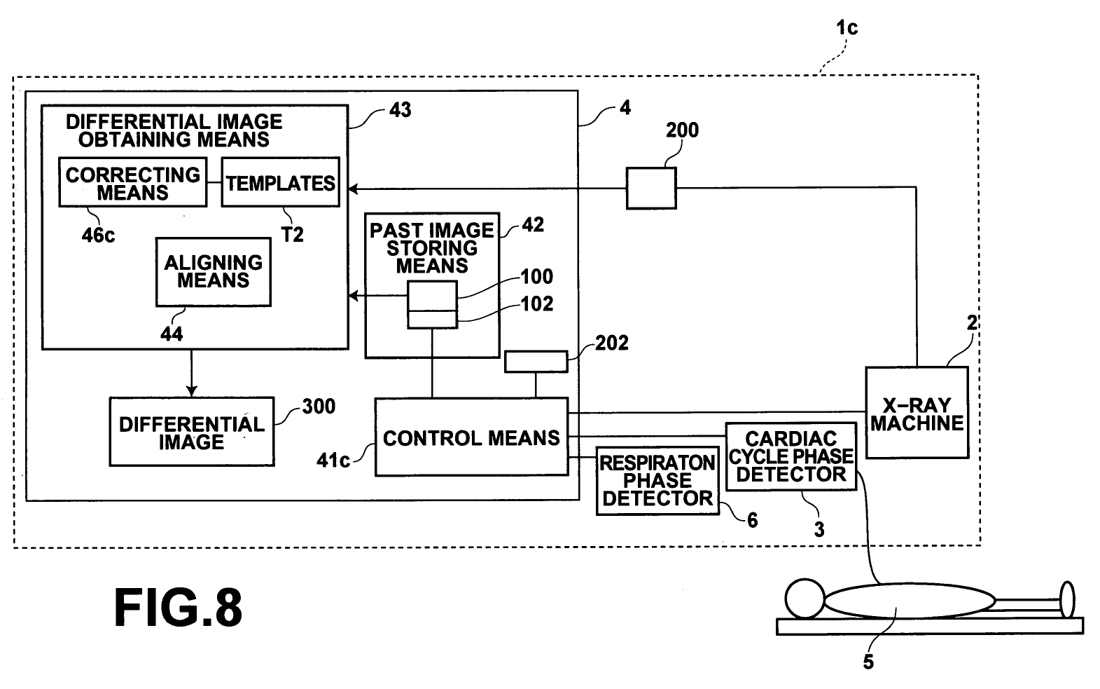

Now, reference is made to FIG. 8. As shown in FIG. 8, a differential image obtaining apparatus 1c according to a fourth embodiment of the present invention comprises the X-ray machine (radiographing means) 2, such as computed radiography (CR) or the like, for X-raying the subject 5; the cardiac cycle phase detector (cardiac cycle phase detecting means) 3 for detecting cardiac cycle phases of the subject 5; the respiration phase detector (respiration phase detecting means) 6 for detecting respiration phases of the subject 5; and the computer 4 for controlling the X-ray machine 2 for the timing of imaging.

The computer 4 has a control means 41c for controlling the X-ray machine 2 for the timing of imaging; the past image storing means for storing the past image 100 obtained by the X-ray machine 2; and the differential image obtaining means 43 for obtaining the differential image 300 from the past image 100 and current image 200. The differential image obtaining means 43 has a correcting means 46c for correcting both the current and past images such that they have the identical respiration phase, and the aligning means 44 for aligning the thoraces between the past and current images.

The control means 41c is connected to the cardiac cycle phase detector 3, and receives cardiac cycle phases of the subject 5 in real time. It outputs the X-ray generation signal to the X-ray machine 2 (current image obtaining means) for instructing it to X-ray the subject 5 when received cardiac cycle phase corresponds to that of the past image, that is, when the current image 200 having the identical cardiac cycle phase to that of the past image is obtained. The control means 41c is also connected to the respiration phase detector 6, and stores the respiration phases at the time when respective images were obtained in the computer as the respiration information 102 and 202 respectively.

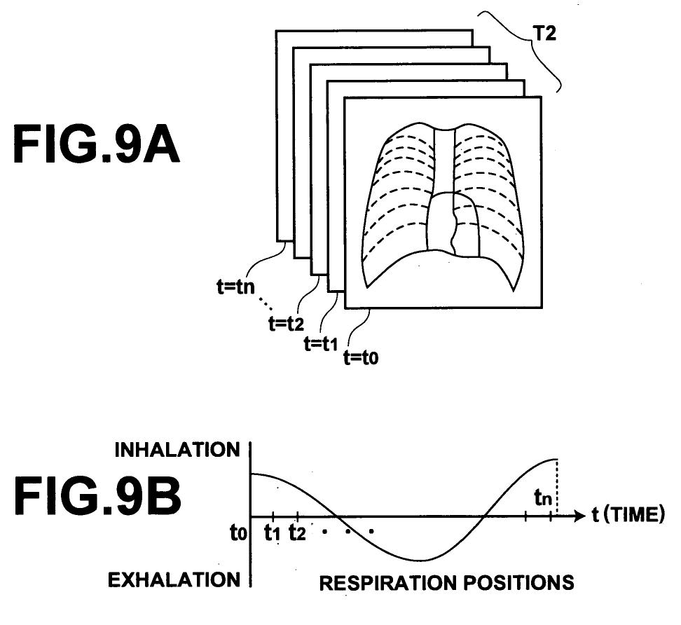

As in the third embodiment, a series of X-ray images of the subject are obtained in advance with a low radiation dosage. This produces a plurality of images having identical cardiac cycle phase but different respiration phases, and templates T2 as shown in FIG. 9A, each corresponding to each of the respiration phases as shown in FIG. 9B are created. Then, the image corrections are performed based on the templates T2.

The ribs and diaphragm will move up and down according to the respiration phase, which will cause the lung fields to be moved. Consequently, each of the regions, such as the lung field regions (Pa, Pb), mediastinal region (Pd), cardiac region (Pc), and the like as shown in FIG. 5, is extracted from each of the templates T2. In addition, ribs are extracted, and the lung fields are warped such that the locations of mediastinal region, ribs, circumference of the lung fields, and bottom region of the lung fields (diaphragm) are aligned.

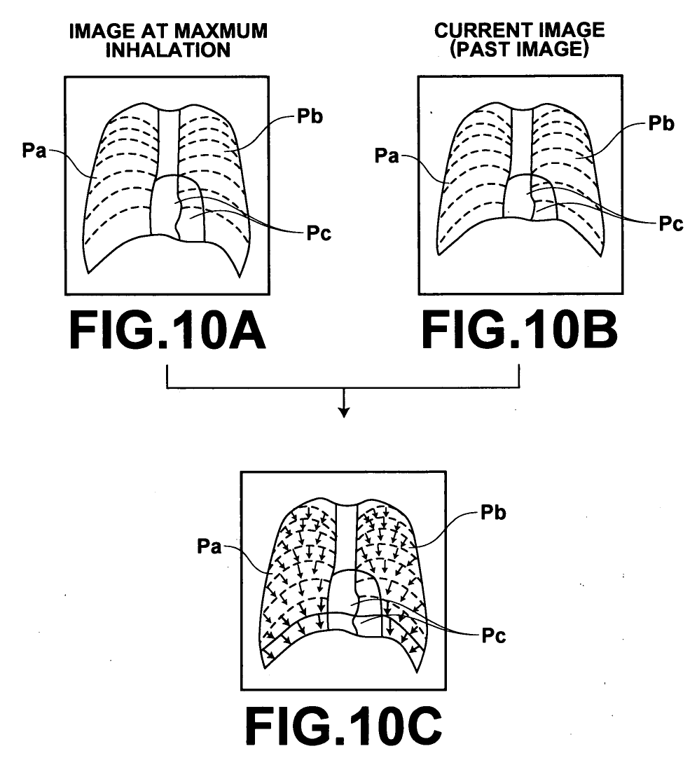

The correcting means 46c extracts each of the regions, such as the lung field regions (Pa, Pb), mediastinal region (Pd), cardiac region (Pc) and the like, as well as the ribs, from the past image 100. It also extracts each of the regions, such as the lung field regions (Pa, Pb), mediastinal region (Pd), cardiac region (Pc) and the like, as well as the ribs, from the current image 200. Then, it warps the pixels within the lung fields of the past image 100 and current image 200 (shown in FIG. 10B) to the positions of the respective templates T2 having lung fields that corresponds to those of the maximum inhalation (FIG. 10A), which has been selected based on the respiration phases included in the respiration information 101 and 201. In the manner described above, the past image 100 and current image 200 are corrected to have the respiration phase of maximum inhalation (FIG. 10C).

As in the embodiments described above, the differential image obtaining means 43 performs the aligning operation (through the aligning means 44) between the past image 100 and the current image 200, which have been corrected to have the respiration phase of maximum inhalation, before obtaining the differential image 300.

As has been described hereinbefore, in the embodiment described above, the past and current images are corrected to have the identical respiration phase. Then, the aligning operation is performed between the past and current images before obtaining the differential image. This allows a pale shadow, such as lung cancer or the like, to be highlighted without being faded by the influence of the respiration (motion artifacts) even in cases where the subject has difficulties to hold the breath at the time of X-raying.

Hereinafter, the differential image obtaining apparatus according to a fifth embodiment of the present invention will be described. In the present embodiment, past and present images obtained with the subject breathing voluntarily are used, and the differential image is obtained after either the pastor current image has been corrected such that both images have the identical cardiac and respiration phases. In the present embodiment, components Identical to those used in the embodiments described above are given the same reference numerals and will not be elaborated upon further here.

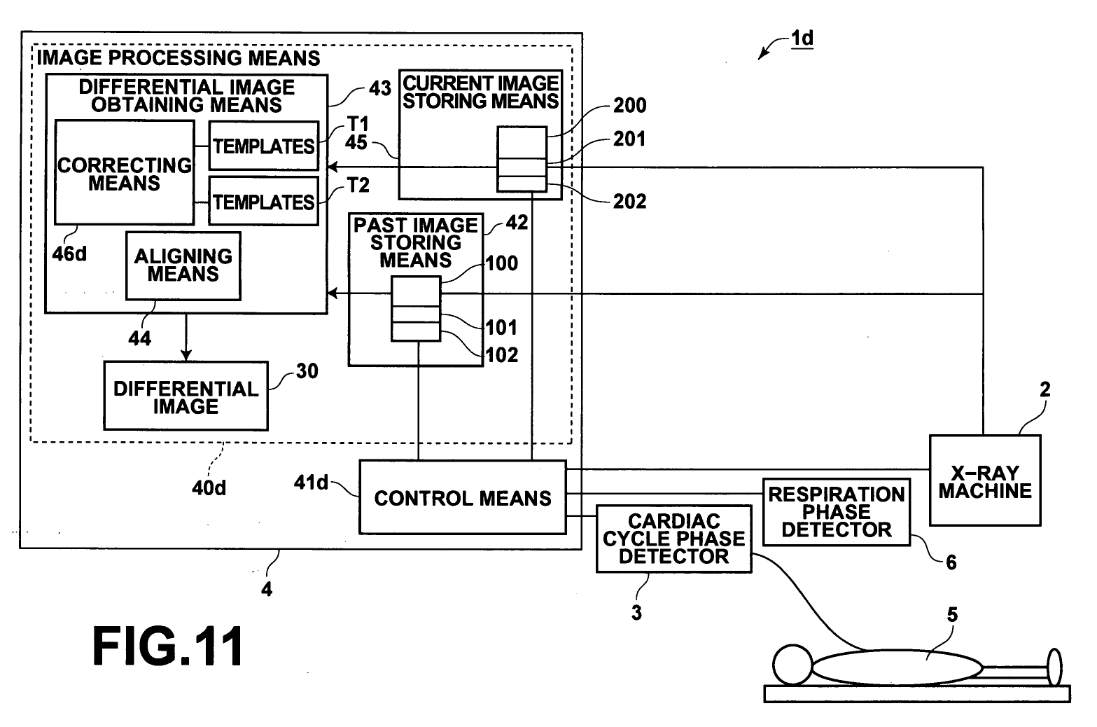

Now, reference is made to FIG. 11. As shown in FIG. 11, a differential image obtaining apparatus 1d comprises the X-ray machine (radiographing means) 2, such as computed radiography (CR) or the like, for X-raying the subject 5; the cardiac cycle phase detector (cardiac cycle phase detecting means) 3 for detecting cardiac cycle phases of the subject 5; the respiration phase detector (respiration phase detecting means) 6 for detecting respiration phases of the subject 5; and the computer 4 for controlling the X-ray machine 2 for the timing of imaging.

The computer 4 has a control means 41d for controlling the X-ray machine 2 for the timing of imaging, and an image processing means 40d for obtaining the differential image from the images obtained by the X-ray machine 2.

The image processing means 40d has the past image storing means 42 for storing the past image 100 obtained by the X-ray machine 2; the current image storing means 45 for storing the current image 200 obtained by the X-ray machine 2; and the differential image obtaining means 43 for obtaining the differential image 300 from the past image 100 and current image 200. The differential image obtaining means 43 has a correcting means 46d for correcting the past image such that it has the identical cardiac and respiration phases to those of the current image, and the aligning means 44 for aligning the thoraces between the past image 100 and current image 200.

The past image storing means 42 stores the past image 100, and the cardiac cycle information 101 that indicate the cardiac cycle phase, and the respiration information 102 that indicates the respiration phase of the subject at the time when the past image 100 was obtained. The current image storing means 45 stores the current image 200, and the cardiac cycle information 201 that indicates the cardiac cycle phase, and the respiration information 202 that indicates the respiration phase of the subject at the time when the past image 100 was obtained. The control means 41d is connected to the cardiac cycle phase detector 3 and respiration phase detector 6, and the cardiac cycle and respiration phases of the subject 5 are inputted to the control means 41d in real time from the cardiac cycle phase detector 3 and respiration phase detector 6 respectively. The cardiac information 101, 201, and the respiration information 201, 202 are recorded data of the cardiac cycle phases and respiration phases at the times when the past image 100 and current image 200 were obtained.

In the present invention, a series of X-ray images of the subject are obtained with a low radiation dosage in advance, as in the second and third embodiments. This produces a plurality of images having the identical respiration phase but different cardiac cycle phases, and a plurality of images having the identical cardiac cycle phase but different respiration phases for creating the templates T1 and T2.

Based on the templates T1 and T2, the correcting means 46d first corrects the past and current images such that both images have the respiration phase of maximum inhalation using the templates T2, as in the fourth embodiment. Then, it corrects either the past image 100 or current image 200, both of which having been corrected to have the respiration phase of maximum inhalation, such that the past and current images have the identical cardiac cycle phase using the templates T1, as in the third embodiment.

Further, the differential image obtaining means 43 performs the aligning operation (through the aligning means 44) between the past image 100 and the current image 200, which have been corrected to have the identical cardiac and respiration phases, before obtaining the differential image 300, as in the embodiments described above.

As has been described hereinbefore, in the present embodiment, either the past or current image is corrected such that both images have the identical cardiac and respiration phases. Then, the aligning operation is performed between the past and current images before obtaining the differential image. This allows a pale shadow, such as lung cancer or the like, to be highlighted without being faded by the influence of the cardiac movement or respiration (motion artifacts).

In the third, forth and fifth embodiments described above, the image corrections are performed using templates provided for individual subjects. In cases where such templates are not provided in advance, lung field regions, mediastinal regions, cardiac regions, and the like at each cardiac cycle phase may be extracted from a multitude of subject images, and the average amount of variation in the position of each pixel within the lung fields is obtained empirically to perform warping. For example, the amount of variation for the circumference of the cardiac region that varies in accordance with cardiac cycle phases may be obtained as the amount of variation in distance from the center of gravity of the cardiac region in advance for image correction.

In the same manner as in the cardiac phase, if templates for respiration phases are not provided in advance, average amount of pixel movement may be obtained from a multitude of subject images for warping.

In the third and fifth embodiments described above, the computer is connected to the X-ray machine, cardiac cycle detector, and the like. But, an alternative arrangement may be made, in which the past image, current image, cardiac cycle information, and the like are read out from a portable storage medium, such as a DVD, without connecting the X-ray machine, cardiac phase detector, and the like to the computer. Further, another alternative arrangement may also be made, in which the computer is connected to a file server through a network, whereby the past and current images, cardiac information, and the like are read out from the file server to the computer.

Still further, in the third, fourth and fifth embodiments, still another alternative arrangement may be made, in which each of the means within the computer may be provided in the X-ray machine.

In each of the embodiments described above, a scattered radiation removing grid may be used to prevent the scattered rays from entering between the subject 5 and the radiation detecting surface, such as an imaging plate, when X-raying the subject by the X-ray machine 2. A typical scattered radiation removing grid comprises a multitude of radiation shielding lead foils superimposed in parallel to the radiation rays irradiated from the X-ray generating source with a gap therebetween. If imaging is performed using such a scattered radiation removing grid, an image having the layer structure of the scattered radiation removing grid superimposed thereon is obtained.

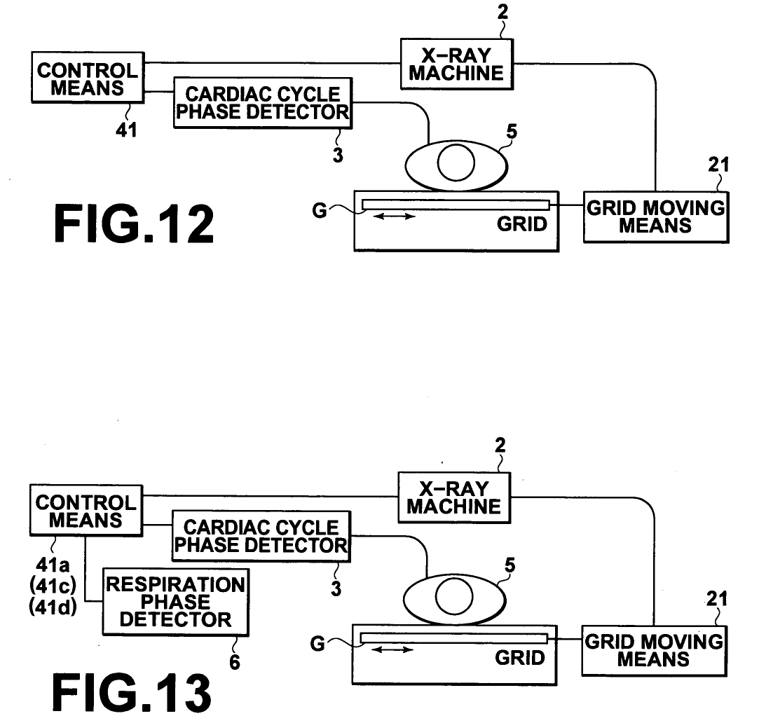

In order to avoid the superimposition described above, the X-ray machine 2 is provided with a grid moving mechanism (grid moving means) 21 for moving a scattered radiation removing grid G in the directions that traverse the grid layer structure in reciprocation while the X-rays are being irradiated to blur the grid image (FIGS. 12 and 13). The grid image becomes more blurred with the travel distance of the scattered radiation removing grid G within the time frame of X-ray irradiation. Therefore, it is desirable that the scattered radiation removing grid G is moved such that it reaches the maximum traveling speed within the time frame of X-ray irradiation. Consequently, control means 41 (41a, 41c, 41d) is connected to the grid moving means 21, and the grid moving means 21 is controlled by the control means 41 such that the scattered radiation removing grid G reaches the maximum traveling speed based on the timing of outputting the X-ray generation signal.

The scattered radiation removing grid G is filled with a material which is transparent to radiation, such as wood or aluminum, in the gap to sustain the physical structure of the grid. The grid moving means 21 moves the scattered radiation removing grid G mechanically, so that the grid can not maintain its maximum traveling speed at the turning-back points. Consequently, in order to control the grid moving means 21 such that the scattered radiation removing grid G reaches the maximum traveling speed when the cardiac phase (or respiration phase) of the subject corresponds to the cardiac phase (or respiration phase) of the timing of imaging, for example, a synchronized signal, which is synchronized with the cardiac phase (or respiration phase) detected by the control means 41 (41a, 41c, 41d), is sent to the grid moving means 21. Alternatively, a grid activation signal for activating the scattered radiation removing grid G may be sent from the control means 41 (41a, 41c, 41d) to the grid moving means 21 such that the scattered radiation removing grid reaches the maximum traveling speed when the cardiac phase (or respiration phase) of the subject corresponds to the cardiac phase (or respiration phase) of the timing of imaging.

If imaging is performed without moving the scattered radiation removing grid G, and an image having the layer structure of the grid superimposed thereon is obtained, a GPR (grid pattern removing) process may by performed on the image to remove the grid pattern, before being inputted to the correcting means or differential image obtaining means described above as the past or current image.

Alternatively, the imaging is performed using a high density grid, instead of moving the grid, to make the grid pattern on the image undistinguishable. Preferably, the grid with the density greater than or equal to around 5 cycle/mm (which is greater than the Nyquist frequency based on the pixel density for reading) is used for general X-ray images, such as chest X-rays or the like. For mammography and the like, the grid with the density greater than or equal to around 10 cycle/mm (which is greater than the Nyquist frequency based on the pixel density for reading) is preferably used. The scattered radiation removing grid may be a high density grid which is less than or equal to the minimum resolution detectable by the detecting unit for detecting X-ray information on the detecting surface, such as an imaging plate, that is, less than the resolution of the current image.

Alternatively, the air-gap method may be used, in which the detecting surface, such as an imaging plate, is placed away from the subject by approximately 15 to 20 cm to remove the scattered radiation scattered from the subject, instead of using the scattered radiation removing grid.

By using the scattered radiation removing grid or placing the detecting surface away from the subject as described above, an image without influence of the scattered radiation may be obtained.

Further, in each of the embodiments described above, the cardiac cycle phase is detected by the cardiac phase detecting means and stored as the cardiac cycle information. The cardiac cycle information may be the information obtained by extracting the cardiac region, and detecting the cardiac cycle phase based on the size and shape of the extracted cardiac region. Likewise, the respiration information may be the information obtained by extracting the thorax region, and detecting the respiration phase based on the size and shape of the thorax region, instead of detecting it through the respiration phase detecting means, and storing it as the respiration information. More specifically, such information may be obtained by the use of the detector, such as an FPD.

The program for executing each of the functions of the computer and X-ray machine may be recorded on a recording medium, such as a CD-ROM, and then installed thereon. Alternatively, it may be installed on the computer through a network.

1. A differential image obtaining method, in which a current image is obtained through X-raying, and a differential image is obtained by obtaining the difference between said current image and a past image provided in advance through X-raying, said method comprising the steps of:
detecting cardiac cycle phases of a subject;
obtaining said current image through X-raying at a time when the cardiac cycle phase detected by said cardiac cycle phase detecting step corresponds to the cardiac cycle phase of said past image; and
obtaining said differential image from said current and past images.

2. A differential image obtaining apparatus, in which a current image is obtained through X-raying, and a differential image is obtained by obtaining the difference between said current image and a past image provided in advance through X-raying, said apparatus comprising:
a detecting means for detecting cardiac cycle phases of said subject;
a current image obtaining means for obtaining said current image through X-raying at a time when the cardiac cycle phase detected by said detecting means corresponds to the cardiac cycle phase of said past image; and
a differential image obtaining means for obtaining said differential image from said current and past images.

3. The differential image obtaining apparatus according to claim 2, wherein said current image obtaining means further comprises:
a scattered radiation removing grid;
a grid moving means for moving said grid; and
a grid control means for controlling said grid moving means such that said grid reaches the maximum traveling speed when said current image is obtained.

4. The differential image obtaining apparatus according to claim 2, wherein said differential image obtaining means further comprises an aligning means for aligning the thoraces between said past and current images.

5. A differential image obtaining apparatus, comprising:
a past image storing means for storing a past image obtained through X-raying the chest of a subject and cardiac cycle information that indicates the cardiac cycle phase of said subject at the time when said past image was obtained;
a radiographing means for X-raying a subject;
a cardiac cycle phase detecting means for detecting cardiac cycle phases of said subject;
a control means for controlling said radiographing means such that a current image is obtained through X-raying the chest of said subject at a time when the cardiac cycle phase detected by said cardiac cycle phase detecting means corresponds to the cardiac cycle phase of said subject at the time when said past image was obtained; and
a differential image obtaining means for obtaining a differential image from said past and current images.

6. The differential image obtaining apparatus according to claim 5, wherein said radiographing means further comprises:
a scattered radiation removing grid; and
a grid moving means for moving said grid, and wherein said control means is configured to further control said grid moving means such that said grid reaches the maximum traveling speed when said current image is obtained.

7. The differential image obtaining apparatus according to claim 5, wherein said differential image obtaining means further comprises an aligning means for aligning the thoraces between said past and current images.

8. A differential image obtaining apparatus, comprising:
a past image storing means for storing a past image obtained through X-raying the chest of a subject, cardiac cycle information that indicates the cardiac cycle phase of said subject at the time when said past image was obtained, and respiration information that indicates the respiration phase of said subject at the time when said past image was obtained;
a radiographing means for X-raying a subject;
a cardiac cycle phase detecting means for detecting cardiac cycle phases of said subject;
a respiration phase detecting means for detecting respiration phases of said subject;
a control means for controlling the radiographing means such that a current image is obtained through X-raying the chest of said subject at a time when the cardiac cycle phase detected by said cardiac cycle phase detecting means corresponds to the cardiac cycle phase of said subject at the time when said past image was obtained, and the respiration phase detected by said respiration phase detecting means corresponds to the respiration phase of said subject at the time when said past image was obtained; and
a differential image obtaining means for obtaining a differential image from said past and current images.

9. The differential image obtaining apparatus according to claim 8, wherein said radiographing means further comprises:
a scattered radiation removing grid; and
a grid moving means for moving said grid, and wherein said control means is configured to further control said grid moving means such that said grid reaches the maximum traveling speed when said current image is obtained.

10. The differential image obtaining apparatus according to claim 8, wherein said differential image obtaining means further comprises an aligning means for aligning the thoraces between said past and current images.

11. A differential image obtaining apparatus, comprising:
a past image storing means for storing a past image obtained through X-raying the chest of a subject, and cardiac cycle information that indicates the cardiac cycle phase of said subject at the time when said past image was obtained;
a radiographing means for X-raying a subject;
a cardiac cycle phase detecting means for detecting the cardiac cycle phase of said subject;
a respiration phase detecting means for detecting the respiration phase of said subject;
a control means for controlling the radiographing means such that a current image is obtained through X-raying the chest of said subject at a time when the cardiac cycle phase detected by said cardiac cycle phase detecting means corresponds to the cardiac cycle phase of said subject at the time when said past image was obtained; and
a correcting means for correcting at least either said past or current image such that said past and current images have the identical respiration phase based on the respiration phase of said past image, and the respiration phase detected by said respiration detecting means at the time when said current image was obtained; and
a differential image obtaining means for obtaining a differential image from said past and current images having the identical respiration phase.

12. The differential image obtaining apparatus according to claim 11, wherein said radiographing means further comprises:
a scattered radiation removing grid; and
a grid moving means for moving said grid, and wherein said control means is configured to further control said grid moving means such that said grid reaches the maximum traveling speed when said current image is obtained.

13. The differential image obtaining apparatus according to claim 11, wherein said differential image obtaining means further comprises an aligning means for aligning the thoraces between said past and current images.

14. A differential image obtaining apparatus, comprising:
a past image storing means for storing a past image obtained through X-raying the chest of a subject, and cardiac cycle information that indicates the cardiac cycle phase of said subject at the time when the past image was obtained;
a current image storing means for storing a current image obtained through X-raying the chest of a subject and cardiac cycle information that indicates the cardiac cycle phase of said subject at the time when said current image was obtained;
a correcting means for correcting at least either said past or current image such that said past and current images have the identical cardiac cycle phase based on the cardiac cycle phase at the time when said past image was obtained, and the cardiac cycle phase at the time when said current image was obtained; and
a differential image obtaining means for obtaining a differential image from the past and current images having the identical cardiac cycle phase.

15. The differential image obtaining apparatus according to claim 14, wherein said differential image obtaining means further comprises an aligning means for aligning the thoraces between said past and current images.

16. A differential image obtaining apparatus, comprising:
a past image storing means for storing a past image obtained through X-raying the chest of a subject, cardiac cycle information that indicates the cardiac cycle phase of the subject at the time when the past image was obtained, and respiration information that indicates the respiration phase of the subject at the time when the past image was obtained;
a current image storing means for storing a current image obtained through X-raying the chest of a subject, cardiac cycle information that indicates the cardiac cycle phase of said subject at the time when said current image was obtained, and respiration information that indicates the respiration phase of said subject at the time when the current image was obtained;
a correcting means for correcting at least either said past or current image such that said past and current images have the identical cardiac cycle and respiration phases based on the cardiac cycle and respiration phases at the time when said past image was obtained, and the cardiac cycle and respiration phases at the time when said current image was obtained; and
a differential image obtaining means for obtaining a differential image from the past and current images having the identical cardiac cycle and respiration phases.

17. The differential image obtaining apparatus according to claim 16, wherein said differential image obtaining means further comprises an aligning means for aligning the thoraces between said past and current images.

18. A differential image obtaining method, comprising:
a past image storing step for storing a past image obtained through X-raying the chest of a subject, and cardiac cycle information that indicates the cardiac cycle phase of said subject at the time when said past image was obtained;
a current image storing step for storing a current image obtained through X-raying the chest of a subject and cardiac cycle information that indicates the cardiac cycle phase of said subject at the time when said current image was obtained;
a correcting step for correcting at least either said past or current image such that said past and current images have the identical cardiac cycle phase based on the cardiac cycle phase at the time when said past image was obtained, and the cardiac cycle phase at the time when said current image was obtained; and
a differential image obtaining step for obtaining a differential image from the past and current images having the identical cardiac cycle phase.

19. A program for causing a computer to execute:
a past image storing step for storing a past image obtained through X-raying the chest of a subject, and cardiac cycle information that indicates the cardiac cycle phase of said subject at the time when said past image was obtained;
a control step for controlling a radiographing means such that a current image is obtained through X-raying the chest of the subject at a time when the cardiac cycle phase detected by a cardiac cycle phase detecting means for detecting cardiac cycle phases of the subject corresponds to the cardiac cycle phase of said subject at the time when said past image was obtained; and
a differential image obtaining step for obtaining a differential image from said past and current images.

20. A program for causing a computer to execute:
a past image storing step for storing a past image obtained through X-raying the chest of a subject, and cardiac cycle information that indicates the cardiac cycle phase of said subject at the time when said past image was obtained;
a current image storing step for storing a current image obtained through X-raying the chest of a subject, and cardiac cycle information that indicates the cardiac cycle phase of said subject at the time when the current image was obtained;
a correcting step for correcting at least either said past or current image such that said past and current images have the identical cardiac cycle phase based on the cardiac cycle phase at the time when said past image was obtained, and the cardiac cycle phase at the time when the current image was obtained; and
a differential image obtaining step for obtaining a differential image from the past and current images having the identical cardiac cycle phase.
